(19) United States
(12) Patent Application Publication
(10) Pub. No.: US 2015/0274770 A1
(43) Pub. Date: Oct. 1, 2015

(54) NOVEL PROCESS AND INTERMEDIATE FOR PREPARATION OF ULIPRISTAL

(71) Applicant: LUPIN LIMITED, Mumbai (IN)

(72) Inventors: Purna Chandra Ray, Pune (IN); Ajinath Tukaram Pathade, Pune (IN); Suryaprakash Pandurang Patil, Pune (IN); Yuvraj Atmaram Chavan, Pune (IN); Girij Pal Singh, Pune (IN); Dnyaneshwar Tukaram Singare, Pune (IN); Yogesh Dadaji Pawar, Pune (IN)

(21) Appl. No.: 14/436,179

(22) PCT Filed: Oct. 1, 2013
(86) PCT No.: PCT/IB2013/059030

§ 371 Date: Apr. 16, 2015

(30) Foreign Application Priority Data
Oct. 18, 2012 (IN) 1188/KOL/2012
Jan. 16, 2013 (IN) 51/KOL/2013

(51) Int. Cl. C07J 41/00

(52) U.S. Cl. CPC C07J 41/0083

(57) ABSTRACT

The present invention is related to a novel process for the preparation of ulipristal (I) that comprises reaction of 17-α-ethynyl-17-β-hydroxy-11-β-(4-N,N-dimethylamino phenyl)- 9-norpregna-4,9-diene-3-one (III) with phenyl sulphenyl chloride (IVa) or p-nitro phenyl sulphenyl chloride (Nb) in the presence of organic base and solvent to give sulfoxide (Va) or (Vb) respectively. Sulfoxides (Va) or (Vb) are reacted with alkali metal alkoxide in alcoholic solvent followed by treatment with aqueous acid. The present invention also relates to novel intermediate 11-β-(4-N,N-dimethylaminophenyl)-21(p- nitro-phenyl-sulphinyl)-19-norpregna-4(5), 9(10), 17(20) 20-tetraene, 3-one (Vb).

### FIELD OF THE INVENTION

The present invention relates to novel process for obtaining ulipristal (I) and a novel intermediate 11 -β-(4-N,N-dimethylaminophenyl)-21(p-nitro-phenyl-sulphinyl)-19-norpregna-4(5), 9(10), 17(20) 20-tetraene, 3-one (Vb).

### BACKGROUND OF THE INVENTION:

Ulipristal is, chemically known as 17-α-hydroxy-11-β-[4-(dimethylamino) phenyl]-19 norpregna-4,9-diene-3,20-dione, represented by formula I. It's acetyl derivative known as ulipristal acetate (II), it possesses anti-progestational and anti-glucocoticoid activity which is useful in therapeutic and contraceptive gynaecological indications.

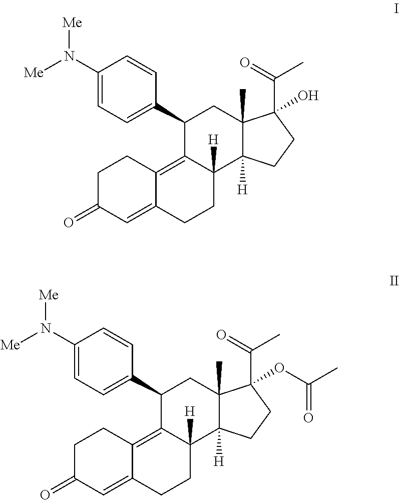

The synthesis of ulipristal is disclosed in patent U.S. Pat. No. 4,954,490 which involves oxidation of 17-vinyl compound to obtain diol compound by using osmium tetroxide as shown below.

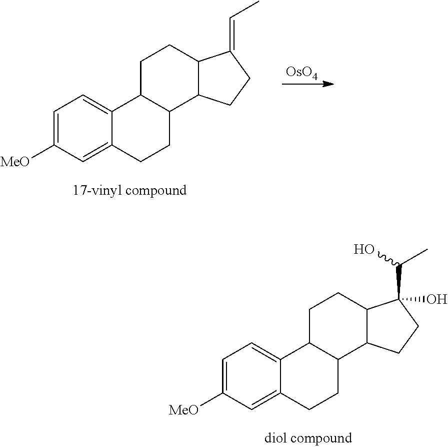

The use of osmium tetroxide makes the process costly and hence industrially non feasible. The patent U.S. Pat. No. 5,929,262 involves the epoxidation of bis-ketal compound, as shown in the reaction below. Later patent application U.S. 2006111577 states that, the epoxidation of the bis-ketal compounds remains incomplete and involves extensive chromatographic separation in order to purify the epoxy product.

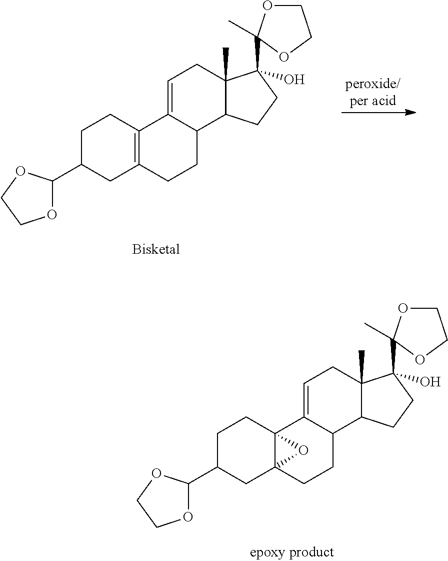

Further, the ulipristal acetate obtained in example 7 of U.S. Pat. No. 5,929,262 is in the form of yellow crystals. The Indian patent application IN 1987/CHENP/2005 states that the yellow color of ulipristal acetate is due to the presence of impurities, mainly phenol compounds which requires further purification and causes decrease in yields. Rao et al, Steroids 63, 1998, pages 53-57 describes preparation of ulipristal via 17-beta nitroxy derivate as an intermediate wherein yield of nitroxy derivative is only 29% and requires purification by column chromatography.

In view of the disadvantages of prior methods, a need exists for a more efficient process for the preparation of ulipristal (I).

### SUMMARY OF THE INVENTION

The present invention relates to a novel process for preparation of ulipristal (I), which comprises: (i) reaction of 3-keto compound (III) with phenyl sulphenyl chloride (IVa) or p-nitro phenyl sulphenyl chloride (IVb) in the presence of organic base and solvent to give sulfoxide (Va) or (Vb) respectively, (ii) reaction of sulfoxide (Va) or (Vb) with alkali metal alkoxide in alcoholic solvent to give 20-alkoxy-compound (VI) and (iii) reaction of 20-alkoxy-compound (VI) with aqueous acid. The present invention also relates to steroidal intermediate 11-β-(4-N,N-dimethylaminophenyl)-21(p-nitro-phenyl-sulphinyl)-19-norpregna-4(5), 9(10), 17(20) 20-tetraene, 3-one (Vb) and process for its preparation. The intermediate (Vb) is useful in the preparation of ulipristal (I).

### DESCRIPTION OF THE DRAWINGS

FIG. 1: X-ray powder diffractogram (XRPD) of 11-β-(4-N,N-dimethylaminophenyl)-21(p-nitro-phenyl-sulphinyl)-19-norpregna-4(5), 9(10), 17(20) 20-tetraene, 3-one (Vb).

### DETAILED DESCRIPTION OF THE INVENTION

The present invention is related to a novel process for the preparation of ulipristal (I), that comprises the following steps:
(i) reaction of 3-keto compound (III) with phenyl sulphenyl chloride (IVa) or p-nitro phenyl sulphenyl chloride (IVb) in the presence of organic base and solvent to give sulfoxide (Va) or (Vb) respectively,
(ii) reaction of sulfoxide (Va) or (Vb) with alkali metal alkoxide in alcoholic solvent to give 20-alkoxy-compound (VI) and
(iii) reaction of 20-alkoxy-compound (VI) with aqueous acid to give ulipristal (I).

The compound 17-α-ethynyl-17-β-hydroxy-11-β-(4-N,N-dimethylamino phenyl)-19-norpregna-4,9-diene-3-one (III) was prepared by acidic hydrolysis of 19-norpregn-9-en-20-yn-3-one, 11-β-(4-N,N-dimethylamino phenyl)-5α, 17β-dihydroxy-cyclic 1,2-ethanediyl ketal which in turn can be prepared by the methods disclosed in prior art documents Mais dale et al, Journal of Labelled Compounds & Radiopharmaceuticals (1995), 36(12), 1199-1203; Weignin et al, Steroids (2006), 71(11-12), 949-954; U.S. Pat. No. 7,678,781 and U.S. Pat. No. 7,671,045.

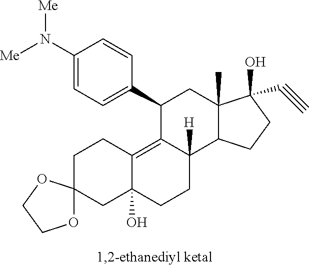

The synthetic scheme of the process for the preparation of ulipristal (I) of present invention is shown in scheme I

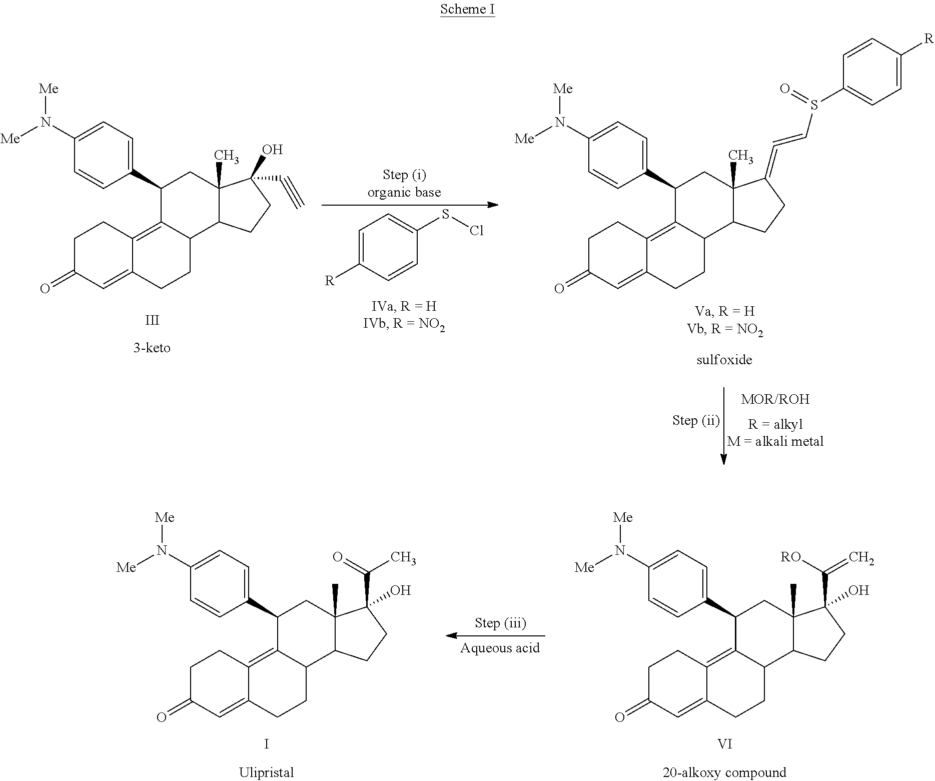

The process of step (i) involves reaction of 3-keto compound (III) with phenyl sulphenyl chloride (IVa) or p-nitro phenyl sulphenyl chloride (IVb) in the presence of organic base and solvent to give sulfoxide Va or Vb respectively.

An organic base that can be used in step (i) includes pyridine, N-methyl morpholine, N-methyl pyrrolidine, tertiary alkyl amine such as triethyl amine, tertiary butyl amine etc, the most preferred base is triethyl amine.

The sulphenyl chloride compounds IVa/IVb are used in the range of 1-10 mole equivalent, preferably 2-4 mole equivalent.

The process of step (i) of the present invention can be carried out in organic solvent that include aromatic hydrocarbons like benzene, toluene and xylene; esters like ethyl acetate and isopropyl acetate; ethers such as ethyl ether, methyl t-butyl ether, di-isopropyl ether and tetrahydrofuran; amides such as formamide, dimethylforamide and N-methyl-pyrrolidone; nitriles such as acetonitrile and propionitrile; chlorinated hydrocarbons such as dichloromethane, ethylene dichloride and chloroform, alcohols such as methanol, ethanol, isopropanol, ketones such as actone, methyl ethyl ketone and mixtures thereof. The most preferred solvent for step (i) is dichloromethane.

The reaction of step (i) is carried at a temperature ranging from −80 to 10° C., more preferably, at −60 to −10° C., most preferably −60 to −40° C.

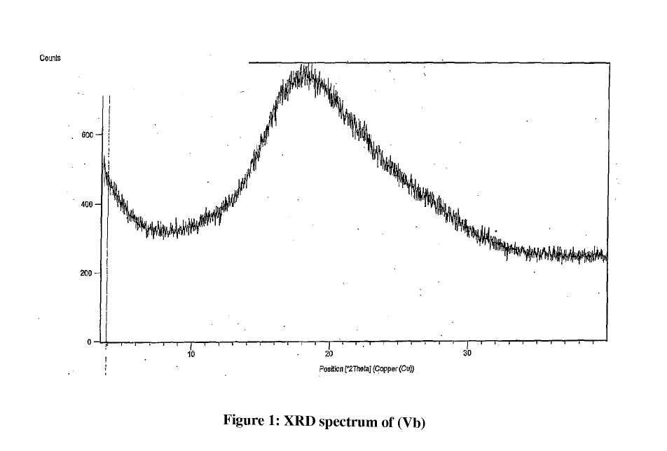

The polymorphic form 11-β-(4-N,N-dimethylaminophenyl)-21(p-nitro-phenyl-sulphinyl)-19-norpregna-4(5), 9(10), 17(20) 20-tetraene, 3-one (Vb) obtained by the process of the present invention is amorphous which is characterized by XRPD pattern as shown in FIG. 1.

The process of step (ii) involves of reaction of sulfoxide (Va or Vb) with alkali metal alkoxide as base in alcoholic solvent to give 20-alkoxy compound (VI, R=C₁-C₅ alkyl). Sodium alkoxide is used in the range of 1-10 mole equivalent, preferably 2-4 mole equivalent.

The alkali metal alkoxide used in step (ii) is selected from a group comprising of sodium methoxide, sodium ethoxide, potassium tertiary butoxide and the like. The most preferred base is sodium methoxide.

The step (ii) can be carried out in C₁-C₅ alcohol such as methanol, ethanol, isopropanol, n-butanol, iso-butanol, t-butanol, pentanol etc, preferably methanol.

The reaction of step (ii) is carried at a temperature ranging from 10 to 100° C., preferrably range is 50-80° C.

The process of step (iii) involves of reaction of 20-alkoxy-compound (VI) with acid in water to give ulipristal (I).

The acid used in step (iii) is selected from group of hydrochloric acid, sulphuric acid, phosphoric acid, methane sulphonic acid, para-toluene sulfonic acid, propionic acid, acetic acid, the most preferred acid is acetic acid.

The reaction of step (iii) is carried at a temperature ranging from 5 to 100° C. The most preferred range is 20-50° C.

Another aspect of the invention involves the conversion of ulipristal (I) to ulipristal acetate (II) using acetic anhydride and perchloric acid. Acetylation of ulipristal (I) to ulipristal acetate (II) can also be carried out by methods known in literature such as U.S. Pat. No. 4,954,490, which discloses acetylation using phosphoric acid and acetic anhydride.

The preferred embodiments of the present invention have been described in the foregoing examples.

### EXAMPLES

The powder X-ray diffraction spectrum is measured using Philips (PAN analytical X'pert pro) diffractogram (percop anti cathode) distance and expressed in terms of inter planar d, Bragg's angle 2 theta, intensity and relative intensity (expressed as a percentage of the most intense peak). The scanning parameters included: measurement range: 3-40 degrees two theta; continuous scan.

The FTIR spectra were obtained using a Perkin-Elmer, Spectrum-100 instrument, using KBr.

The [1H]-NMR were obtained using Bruker, AVANCE II 400 using CDCl₃.

### Example 1

### 17-α-ethynyl-17-β-hydroxy-11-β-(4-N,N-dimethylamino phenyl)-19-norpregna-4,9-diene-3-one (III)

19-Norpregn-9-en-20-yn-3-one, 11-β-(4-N,N-dimethylamino phenyl)-5α, 17β-dihydroxy-cyclic 1,2-ethanediyl ketal (30 gm) was dissolved in water (150 ml) followed by addition of 1:1 aqueous hydrochloric acid (30 ml). The reaction mixture was stirred at 25° C. Dichloromethane (150 ml) was added to the reaction mass followed by addition of 20% sodium hydroxide solution. The reaction mass was stirred at 25° C. for 20 minutes. Organic layer was separated and concentrated. The solid was dried under reduced pressure. Yield=22 gm (83%).

### Example 2

### Preparation of Sulfoxide Compound (Va)

3-keto compound (III, 20 gm) was dissolved in dichloromethane (1000 ml). Triethyl amine (19.5 gm) was added to the reaction mass at 25° C. The reaction was cooled to −60° C. Solution of phenyl sulfynyl chloride (IVa, 28 gm) in dichloromethane (200 ml) was added to the reaction mass. The reaction mixture was stirred. After completion of reaction 1:1 mixture of water-methanol (200 ml) was added. The organic layer was separated, concentrated and residue was chromatographed over silica gel, sulphoxide compound (Va) was eluted with 1:1 ethyl acetate-hexane mixture. The fractions were collected and solvent was distilled out. To the sticky residue hexane was added and distilled. The solid was dried under reduced pressure. Yield: 18 gm (71%).

### Example 3

### Preparation of Sulfoxide Compound (Vb)

The 3-keto compound (III, 1 gm) was dissolved in dichloromethane (10 ml). Triethyl amine (2 ml) and 4-nitro-phenyl sulfynyl chloride solution (IVb, 1.5 gm) was added to the reaction mass. The reaction mixture was stirred at 25-30° C. for 30 minutes. To the reaction 1:1 mixture of water-methanol (200 ml) was added. The organic layer was separated and concentrated. Residue was chromatographed over silica gel. The sulfoxide compound (Vb) was eluted with 1:1 ethyl acetate-hexane mixture. The fractions were collected and distilled. To the sticky residue hexane was added and distilled. The solid was dried under reduced pressure. Yield: 0.70 gm (51%).

### Example 4

### Preparation of Sulfoxide Compound (Vb)

The 3-keto compound (III, 1 gm) was dissolved in dichloromethane (30 ml). Triethyl amine (8.21 gm) and 4-nitro-phenyl sulfiynyl chloride solution (IV, 2.73 gm in 15 ml dichloromethane) was added to the reaction mass. The reaction mixture was stirred at 25-30° C. for 30 minutes. To the reaction 1:1 mixture of water-methanol (30 ml) was added. The organic layer was separated and concentrated. To the residue cyclohexane (60 ml) was added and distilled. The solid was dried under reduced pressure. Yield: 3.2 gm (73%).
MP: 169-169.5° C.
IR: 3092, 2924, 2854, 1946, 1659, 1602, 1519, 1471, 1450, 1378, 1342, 1307, 1234, 1051, 1079, 1051, 1010, 912, 818, 736, 681, 530, 424 cm⁻¹.
MS (m/z): 569.40 [M+1].
[1H]-NMR(CDCl₃): δ8.0 (2H, —CH, dd, J=8.4 and 9.2 Hz), δ7.90 (2H, —CH, dd, J=6.8 and 7.2 Hz), δ7.1(2H, —CH, dd, J=8.4 and 7.2 Hz), δ6.8 (2H, —CH, dd, J=7.6 and 8.4 Hz), δ6.29 (1H, s, —CH), δ5.86 (1H, s, —CH), δ4.4(1H, —CH, dd, J=6.8 and 9.2 Hz), δ3.05(3H, s, —CH₃), δ3.8(6H, s, —CH₃), δ1.0-2.7(16H, m, —CH₂), δ0.8(3H, s, —CH₃)

### Example 5

### Preparation of 20-Methoxy-Compound (VI, R=CH₃) From Sulfoxide Compound (Va)

The sulfoxide compound (Va, 10 gm) was added to methanol (300 ml) followed by addition of sodium methoxide (2 gm). The reaction mass was heated to 64° C. Second lot of sodium methoxide (1 gm) was added to the reaction mass. The reaction was stirred for 6 hours at 65° C. The reaction mass was cooled, silica gel (30 gm) was added to the reaction mass and solvent was distilled out. Residue left was chromatographed over silica gel using 10% ethyl acetate-hexane mixture. The fractions were collected and concentrated under vacuum to give a gummy mass. Yield: 5.6 gm (65%).

### Example 6

### Preparation of 20-Methoxy-Compound (VI, R=CH₃) From Sulfoxide Compound (Vb)

The sulphoxide compound (Vb) was converted to 20-methoxy-compound (VI, R═CH₃) by similar process as above.

### Example 7

### Preparation of Ulipristal (I)

1:1 Mixture of acetic acid-water (35 ml) was added to 20-methoxy-compound (VI, R═CH₃) (3.5 gm). The reaction mass was stirred for one hour at 25° C. Dichloromethane (35 ml) was added to the reaction mixture followed by addition of water (35 ml). The reaction mass was stirred at 25° C. for one hour. The dichloromethane layer was separated and washed with 5% sodium bicarbonate solution. The organic layer was separated and concentrated. To the residue cyclohexane was added and distilled. The solid separated was dried under reduced pressure. Yield: 0.71 gm (74%).

### Example 8

### Preparation of Ulipristal Acetate (II)

Acetic anhydride (2 gm) was added to perchloric acid (0.5 gm) and the mixture was cooled to -20° C. To the reaction mass ulipristal (1 gm) was added and the mixture was stirred for 30 minutes at 0-5° C. To the reaction mass 5% sodium bicarbonate solution was added and warmed to 25° C. The reaction was stirred for thirty minutes and the layers were separated. Organic layer was concentrated and cyclohexane (25 ml) was added. The solvent was distilled and the reaction mass was stirred. The solid was washed with cyclohexane and dried under reduced pressure.

1. A process for the preparation of ulipristal (I) 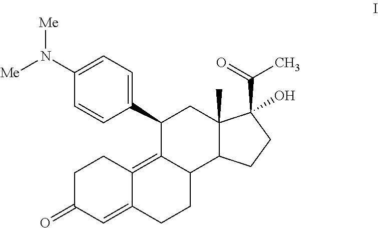 [SMILES: CC(=O)[C@@]1(O)CCC2C3CCC4=CC(=O)CCC4=C3[C@@H](c3ccc(N(C)C)cc3)C[C@@]21C]
comprising
(i) reaction of 3-keto compound (III) 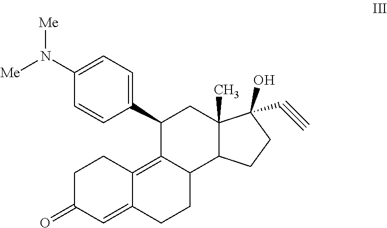 [SMILES: C#C[C@]1(O)CCC2C3CCC4=CC(=O)CCC4=C3[C@@H](c3ccc(N(C)C)cc3)C[C@@]21C]
with phenyl sulphenyl chloride (IVa) or p-nitro phenyl sulphenyl chloride (IVb) 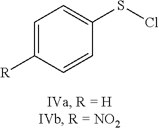
in the presence of organic base and solvent to give sulfoxide (Va) or (Vb) respectively, 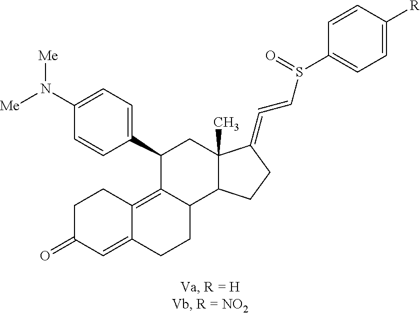
(ii) reaction of sulfoxide (Va) or (Vb) with alkali metal alkoxide in alcoholic solvent to give 20-alkoxy-compound (VI) 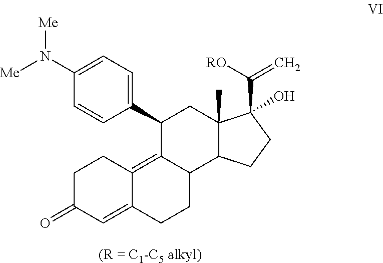
and
(iii) reaction of 20-alkoxy-compound (VI) with aqueous acid.

2. A process of claim 1, wherein organic base used in step (i) is selected from a group comprising of pyridine, N-methyl morpholine, N-methyl pyrrolidine, tertiary alkyl amine such as triethyl amine, tertiary butyl amine.

3. A process of claim 2, wherein the most preferred base is triethyl amine.

4. A process of claim 1, wherein the solvents used in step (i) is selected from group comprising of aromatic hydrocarbons like benzene, toluene and xylene; esters like ethyl acetate and isopropyl acetate; ethers such as ethyl ether, methyl t-butyl 3 ether, di-isopropyl ether and tetrahydrofuran; amides such as formamide, dimethylforamide and N-methyl-pyrrolidone; nitriles such as acetonitrile and propionitrile; chlorinated hydrocarbons such as dichloromethane, ethylene dichloride and chloroform, alcohols such as methanol, ethanol, isopropanol, ketones such as actone, methyl ethyl ketone and mixtures thereof.

5. A process according to claim 4, wherein preferred solvent is dichloromethane.

6. A process of claim 1, wherein alkali metal alkoxide used in step (ii) is selected from sodium methoxide, sodium ethoxide, potassium tertiary butoxide.

7. A process of claim 6, wherein the most preferred alkali metal alkoxide is sodium methoxide.

8. A process of claim 1, wherein alcoholic solvent used in step (ii) is selected from a group comprising of methanol, ethanol, isopropanol, t-butanol, n-butanol, isobutanol, pentanol.

9. A process of claim 8, wherein preferred solvent is methanol.

10. A process of claim 1, wherein acid used in step (iii) is selected from group of hydrochloric acid, sulphuric acid, phosphoric acid, para-toluene sulfonic acid, methane sulfonic acid, propionic acid and acetic acid.

11. A process of claim 10, wherein the most preferred acid is acetic acid.

12. The process of claim 1, further comprising conversion of ulipristal (I) to ulipristal acetate (II).

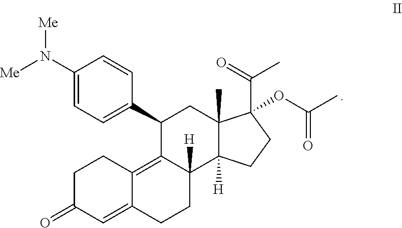

13. 11-β-(4-N,N-dimethylaminophenyl)-21(p-nitro-phenyl-sulphinyl)-19-norpregna-4(5), 9(10), 17(20) 20-tetraene, 3-one (Vb).

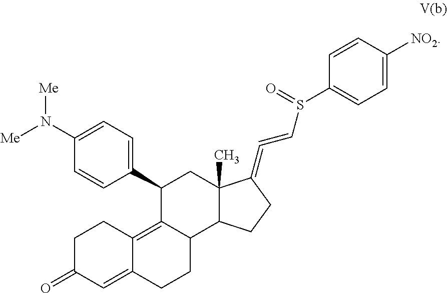 [SMILES: CN(C)c1ccc([C@H]2C[C@]3(C)C(=C=CS(=O)c4ccc([N+](=O)[O-])cc4)CCC3C3CCC4=CC(=O)CCC4=C32)cc1]

14. Compound of claim 13, in amorphous form.

15. Amorphous compound of claim 14, characterized by XRPD pattern as shown in FIG. 1.

16. Amorphous compound of claim 14, characterized by IR peak values at 3092, 2924, 2854, 1946, 1659, 1602, 1519, 1471, 1450, 1378, 1342, 1307, 1234, 1051, 1079, 1051, 1010, 912, 818, 736, 681, 530, 424 cm⁻¹
